(19) United States
(12) Patent Application Publication
(10) Pub. No.: US 2012/0251462 A1
(43) Pub. Date: Oct. 4, 2012

(54) Agent For Smoker Dehabituation

(75) Inventors: Ann-Kathrin Trierweiler, Monchengladbach (DE); Bernhard Mueller, Hurth (DE)

(73) Assignee: Cognis IP Management GmbH, Dusseldorf (DE)

(21) Appl. No.: 13/437,444

(22) Filed: Apr. 2, 2012

Related U.S. Application Data
(60) Provisional application No. 61/471,239, filed on Apr. 4, 2011.

(51) Int. Cl. A61K 31/216; A61P 25/34; A61K 9/68

(52) U.S. Cl. 424/48; 514/533

(57) ABSTRACT

The present invention relates to rosmarinic acid for use in a method for smoker dehabituation, wherein the rosmarinic acid is preferably in the form of a dried, aqueous extract of leaves of lemon balm (Melissa officinalis). In addition, the present invention relates to foods and food supplements for use in a method for smoker dehabituation, wherein these foods and food supplements contain rosmarinic acid, in particular rosmarinic acid in the form of a dried, aqueous extract of leaves of lemon balm (Melissa officinalis).

### CROSS-REFERENCE TO RELATED APPLICATIONS

This application claims priority under 35 USC §119(e) of U.S. Provisional Application No. 61/471,239, filed Apr. 4, 2011.

### FIELD OF THE INVENTION

The present invention relates to rosmarinic acid for use in a method for smoker dehabituation, wherein the rosmarinic acid is preferably in the form of a dried, aqueous extract of leaves of lemon balm (Melissa officinalis). In addition, the present invention relates to foods and food supplements for use in a method for smoker dehabituation, wherein these foods and food supplements contain rosmarinic acid, in particular rosmarinic acid in the form of a dried, aqueous extract of leaves of lemon balm (Melissa officinalis). The food or food supplement containing rosmarinic acid may be in the form of chewing gum, a sucking sweet or a pastille. In particular embodiments, each individual chewing gum or each individual sucking sweet or each individual pastille contains at least 5 mg, at least 10 mg, or at least 20 mg of rosmarinic acid.

### BACKGROUND OF THE INVENTION

The structure of rosmarinic acid may be found, for example, in the online chemistry lexicon Römpp online, Version 3.10.

WO 2009/056208 discloses that extracts of Melissa officinalis (lemon balm) have beneficial effects on cognitive performance. In particular, factors such as stress symptoms are reduced and memory capacity and powers of concentration are increased.

It is an object of the present invention to provide an agent for smoker dehabituation.

This object is achieved by providing rosmarinic acid for use in a method for smoker dehabituation.

### BRIEF SUMMARY OF THE INVENTION

This method for smoker dehabituation is a method for treatment of the human body by therapy within the meaning of Article 53(c) of the European Patent Convention. This therapeutic treatment can be a medical treatment. In this case, the rosmarinic acid used or the agent used which contains the rosmarinic acid is a medicament. This therapeutic treatment can also be a non-medical treatment. In this case, the rosmarinic acid used or the agent used which contains the rosmarinic acid is, for example, a food (also termed functional food) or a food supplement.

The subjects of the subclaims are preferred embodiments of the present invention.

The extracts according to the invention can still contain the extraction medium, e.g. water, or the extraction medium can have been removed, and so the extracts can be, e.g., in the form of dry powder.

### DETAILED DESCRIPTION OF THE INVENTION

Rosmarinic acid in the form of a dried, pulverulent, aqueous extract of leaves of lemon balm (Melissa officinalis) is commercially available from Cognis GmbH from Monheim in Germany under the name Plantalin® Lemon Balm. Cognis GmbH is a business of the BASF Group. Rosmarinic acid may also be extracted from rosemary, thyme, oregano, savory, peppermint and sage.

Nicotine is a substance having stimulating and at the same time calming actions on the nervous system. Nicotine in addition is responsible for the highly additive action of tobacco consumption. The binding of nicotine to nicotinic acetylcholine (nACh) receptors in specific regions of the brain causes the secretion of various neurotransmitters which, inter alia, cause desired calming effects and also increased alertness. The binding of nicotine to the nACh receptors activates the dopaminergic reward system. When the level of active substances in the reward center decreases, the demand for a new dose of nicotine occurs. Addiction and withdrawal symptoms are caused by the constant stimulation of the reward system by the nicotine failing to occur, or the effect lasting for less time.

Tobacco consumption very frequently leads to diseases which affect the heart and lungs, but also other organs of the human body. Tobacco consumption is a high risk factor for heart attack, stroke, chronic obstructive lung disease and cancer, in particular lung cancer, throat and mouth tumors such as, e.g., laryngeal cancer, or else pancreatic cancer. Furthermore, tobacco consumption also participates in the formation of peripheral arteriosclerotic vascular disease and hypertension, depending on the duration and amount of consumption.

When a smoker stops smoking, nicotine receptors are no longer occupied by nicotine and withdrawal symptoms such as disquiet and irritability and also an increased demand for nicotine can occur.

Various known products which are used for smoker dehabituation imitate the mechanism of action of nicotine and thus achieve the same desired effects as the smoking of cigarettes and other tobacco products.

Products such as nicotine patches, nicotine-comprising chewing gums or inhalers ensure that by a dose-controlled supply of nicotine, withdrawal symptoms are ameliorated to abolished, and that the smoker is thus supported in the dehabituation therapy.

In contrast to tobacco consumption, these nicotine preparations do not cause addiction, since they do not give rise to a sudden nicotine rise in the body, but cause a constant low nicotine level.

In vitro tests with rosmarinic acid, which was in the form of a dried, aqueous extract of lemon balm leaves, using what is termed the microelectrode array (MEA) neurochip technology have found that rosmarinic acid has a similar mechanism of action on the nACh receptor as nicotine. Therefore, it is possible that rosmarinic acid is used for smoker dehabituation and in this process acts like nicotine, but without causing the health-damaging effects of tobacco consumption and tobacco smoke.

By binding to the nACh receptor, rosmarinic acid imitates the binding of nicotine and thereby achieves the same effects which are also caused by nicotine, but without causing unwanted health-damaging effects which are caused by various components of tobacco smoke which are formed on the burning of tobacco.

Therefore, rosmarinic acid can produce agonistic effects on the nACh receptor, similar to nicotine, and thereby reduce the demand for nicotine and tobacco consumption. On the basis of the described mechanism of action, rosmarinic acid can therefore replace nicotine and serve for smoker dehabituation for people who wish to give up smoking.

### EXAMPLES

In vitro study

The study described hereinafter was carried out in order to analyze the mechanism of action of rosmarinic acid in the frontal cortex. The rosmarinic acid was in the form of a dried, pulverulent, aqueous extract of leaves of lemon balm (Melissa officinalis). The product Plantalin® Lemon Balm was used. This is commercially available from Cognis GmbH from Monheim in Germany.

The MEA neurochip technology was used in order to study the acute concentration-dependent and receptor-specific effect of lemon balm on the neuronal network activity. For this purpose the substance was tested in acute concentration series in order to characterize the effect thereof on the spontaneous activity of neuronal networks of the frontal cortex. For this test series, in the active range, concentration series were measured in vitro by means of electrophysiological multichannel recordings. Then, the activity pattern of lemon balm was compared with the activity patterns of other characterized substances such as plant extracts and pharmacological active ingredients.

Method

For a nerve cell culture, tissue from the frontal cortex of fetal mice was used. After preparation of the tissue, this was dissociated enzymatically and mechanically. The cell suspension was then seeded onto the pretreated surfaces of the neurochips used. The cells growing directly on the neurochips under controlled conditions developed to form natural nerve networks in the course of 4 to 6 weeks. These nerve networks were comparable to the original tissue.

Since neuronal network cultures develop spontaneous activity in vitro and exhibit pronounced signal patterns which react highly sensitively to their chemical “environment”, they can also be used as a reliable test system for rapid determination of neuroactive and neurotoxic compounds. Electrodes of the chips and the nerve cells are coupled, whereby the action potentials of the cells can be recorded and their amplitude and the electrical activity pattern can be evaluated. This technology of biosystem technology studies electrical activity patterns of neuronal networks on multielectrode arrays (MEA neurochips) during functioning thereof. In this system cells or tissue grow directly on the chip surface and communicate via chemical and electrical signals. The MEA neurochip permits non-invasive long-term measurement of electrical signal patterns of the primary neuronal networks of a multiplicity of recording sites. The MEA neurochip technology enables the characterization of the activity patterns of the networks at the level of the single-cell action potentials and also at the level of complex neuronal networks, the fundamental functional units.

Spontaneously active, single-layer networks on MEA neurochips offer the great possibility of studying the development of neuronal networks and also the generation of time-space patterns of action potentials.

Likewise, they can be used as test systems for studying neurophysiological properties of substances which are technically very difficult to obtain in vivo, or not at all. Despite the complexity, the neurophysiological activity profiles of the neuroactive substances are sensitive and selective and also robust and stable. This makes it possible to prepare precise pharmacological “fingerprints” of neuroactive substances.

The complex description of the changes owing to the treatment of a neuronal network in co-culture with glia permits a differentiated approach in order to quantify the complex effects of neuroactive substances, unknown substances, and complex mixtures.

The concentration range extends from 10 fg/ml to 1 mg/ml and was applied in 10 concentrations cumulatively to the neurochip. After determining the reference activity (native spontaneous activity for 1 h), for each concentration at least 60 min of activity were recorded for the respective concentrations. The network reaction (spike rate) was observed online; the next application proceeded after a stable activity phase of at least 30 min. Over the entire time period, the activity changes of the neuronal network were measured. This test and was repeated in triplicate.

This analysis provides multiparametric information on the changes of neuronal activity in order to be able to judge the therapeutic potential of neuroactive substances. It can also be shown that each substance has its own fingerprint, i.e. how it changes the network activity pattern in a specific manner. Using a substance database, in this manner unknown novel substances can rapidly be compared, which provides indications of possible mechanisms of action or the applicability of the substances as therapy for special indications.

In addition, in the present study, targeted receptor activation or receptor blockade was used in order to demonstrate clear effects. Subsequently, the activity pattern of lemon balm was compared with the activity patterns of other characteristic substances such as plant extracts and pharmacological active ingredients.

Results

Changes in the activity in the frontal cortex which were caused by accumulating concentrations of lemon balm showed the greatest similarity to the activity pattern of the nACh receptor agonist epibatidin. The concentration-dependent assignment of the similarities of activity pattern changes by the classification showed a to epibatidin particularly in the clinically active range.

Epibatidin is an alkaloid having a bicyclic structure obtained from the skin gland secretion of the tricolor poison dart frog (Epipedobates tricolor). It occurs in an amount of less than 1 mg per frog. Epibatidin exceeds the affinity of nicotine for nACh receptors by 120 fold. Epibatidin is a potent nACh receptor agonist and acts on both subtypes of the receptor, the α4β2 and the α3β4 receptor. Results of the study showed, in the clinically relevant range, a similarity of lemon balm to epibatidin of 42-56%.

Other scientists have shown that epibatidin has a similar pharmacological profile to varenicline, a medicament which is used for smoker dehabituation. However, epibatidin is markedly more potent than varenicline. However, epibatidin itself is not used for smoker dehabituation since it is too toxic for use in humans. varenicline is a partial nicotinergic agonist, and binds to nACh receptors and thereby prevents the binding of nicotine. The binding stimulates the receptor and reduces withdrawal symptoms during smoker dehabituation. In particular, the dopaminergic reward system modulated by nACh receptors is blocked in this case and therefore the demand for renewed nicotine supply is reduced. The efficacy of varenicline has been demonstrated in a plurality of randomized clinical studies.

The results of the study with lemon balm showed a mechanism of action similar to that of nicotine. When substances other than nicotine bind to nACh receptors, it can be that less dopamine is secreted. The comparatively low secretion of the reward substance dopamine is sufficient for reducing the demand for nicotine and withdrawal symptoms. Accordingly, lemon balm, owing to the high affinity for the nACh receptor, is suitable as an agent for smoker dehabituation.

1. A method for smoker dehabituation comprising administering rosmarinic acid to a subject in need of smoker dehabituation, thereby reducing demand for nicotine and tobacco consumption in the subject.

2. The method of claim 1, wherein the rosmarinic acid is in the form of an extract obtained from a plant or a plant part which contains rosmarinic acid.

3. The method of claim 2, wherein the extract is an aqueous extract.

4. The method of claim 3, wherein the extract is in the form of a dried powder.

5. The method of claim 2, wherein the plant is selected from the group consisting of rosemary, lemon balm (Melissa officinalis), thyme, oregano, savory, peppermint and sage.

6. The method of claim 3, wherein the extract is an aqueous extract of lemon balm (Melissa officinalis).

7. The method of claim 2, wherein the extract contains at least 0.5% by weight of rosmarinic acid.

8. The method of claim 7, wherein the extract contains at least 1% by weight of rosmarinic acid.

9. The method of claim 8, wherein the extract contains at least 2% by weight of rosmarinic acid.

10. The method of claim 2, wherein the plant is lemon balm (Melissa officinalis).

11. The method of claim 10, wherein the leaves of lemon balm are extracted.

12. The method of claim 1, wherein the rosmarinic acid is incorporated into a food or food supplement.

13. The method of claim 12, wherein the rosmarinic acid incorporated into the food or food supplement is in the form of an extract obtained from a plant or a plant part which contains rosmarinic acid.

14. The method of claim 12, wherein the food or food supplement is selected from the group consisting of chewing gum, a sucking sweet and a pastille.

15. The method of claim 12, wherein the food or food supplement is selected from the group consisting of chewing gum, a sucking sweet and a pastille.

16. The method of claim 15, wherein each individual chewing gum or each individual sucking sweet or each individual pastille contains at least 5 mg, of rosmarinic acid.

17. The method of claim 16, wherein each individual chewing gum or each individual sucking sweet or each individual pastille contains at least 10 mg of rosmarinic acid.

18. The method of claim 17, wherein each individual chewing gum or each individual sucking sweet or each individual pastille contains at least 20 mg of rosmarinic acid.
